SEQUENCE LISTING (41 sequences)

SEQ ID NO: 1 - DNA, 18 bp, Artificial Sequence
  Description of Artificial Sequence Primer
ttgtacacaccgcccgtc

SEQ ID NO: 2 - DNA, 22 bp, Artificial Sequence
  Description of Artificial Sequence Primer
tacggnnaccttgttacgactt

SEQ ID NO: 3 - DNA, 26 bp, Artificial Sequence
  Description of Artificial Sequence Synthetic probe
cggtggggtaaccttttggagccagc

SEQ ID NO: 4 - DNA, 23 bp, Artificial Sequence
  Description of Artificial Sequence Synthetic probe
cggtggcccaaccccttgtggga

SEQ ID NO: 5 - DNA, 30 bp, Artificial Sequence
  Description of Artificial Sequence Synthetic probe
tgggagtgggttgcaaaagaagtaggtagc

SEQ ID NO: 6 - DNA, 30 bp, Artificial Sequence
  Description of Artificial Sequence Synthetic probe
aagtcgtaacaaggtttccgtaggtgaacc

SEQ ID NO: 7 - DNA, 24 bp, Artificial Sequence
  Description of Artificial Sequence Synthetic probe
cgaagtcgttaccttaaccgcaag

SEQ ID NO: 8 - DNA, 27 bp, Artificial Sequence
  Description of Artificial Sequence Synthetic probe
cggtggggtaacctttwtggagccagc

SEQ ID NO: 9 - DNA, 20 bp, Artificial Sequence
  Description of Artificial Sequence Synthetic probe
tccggtgaagtgttcggatc

SEQ ID NO: 10 - DNA, 31 bp, Artificial Sequence
  Description of Artificial Sequence Synthetic probe
cgtacgtaacacccgaagncggtggcgtacg

SEQ ID NO: 11 - DNA, 29 bp, Artificial Sequence
  Description of Artificial Sequence Synthetic probe
acaccacgagagttngtaacacccgaagt

SEQ ID NO: 12 - DNA, 20 bp, Artificial Sequence
  Description of Artificial Sequence Synthetic probe
acccgaaggcgctgcgctaa

SEQ ID NO: 13 - DNA, 24 bp, Artificial Sequence
  Description of Artificial Sequence Synthetic probe
caaacttggtcatttagaggaagt

SEQ ID NO: 14 - DNA, 20 bp, Artificial Sequence
  Description of Artificial Sequence Synthetic probe
tgaggccttcggactggctc

SEQ ID NO: 15 - DNA, 29 bp, Artificial Sequence
  Description of Artificial Sequence Synthetic probe
ttaaccttcgggagggcgcttaccacttt

SEQ ID NO: 16 - DNA, 25 bp, Artificial Sequence
  Description of Artificial Sequence Synthetic probe
cgattgggtgtgctggtgaagtgtt

SEQ ID NO: 17 - DNA, 29 bp, Artificial Sequence
  Description of Artificial Sequence Synthetic probe
cgaggtaacacccgaagncggtggcctcg

SEQ ID NO: 18 - DNA, 34 bp, Artificial Sequence
  Description of Artificial Sequence Synthetic probe
cgcaccacgaaagttngtaacacccgaaggtgcg

SEQ ID NO: 19 - DNA, 21 bp, Artificial Sequence
  Description of Artificial Sequence Synthetic probe
atccggtgaagtgttcggatc

SEQ ID NO: 20 - DNA, 28 bp, Artificial Sequence
  Description of Artificial Sequence Synthetic probe
cttgcgaagtcgttaccttaaccgcaag

SEQ ID NO: 21 - DNA, 25 bp, Artificial Sequence
  Description of Artificial Sequence Synthetic probe
cgtgaggccttcggactggctcacg

SEQ ID NO: 22 - DNA, 31 bp, Artificial Sequence
  Description of Artificial Sequence Synthetic probe
cgcaaacttggtcatttagaggaagtttgcg

SEQ ID NO: 23 - DNA, 24 bp, Artificial Sequence
  Description of Artificial Sequence Synthetic probe
ccggcaacgaccacccagggccgg

SEQ ID NO: 24 - DNA, 37 bp, Artificial Sequence
  Description of Artificial Sequence Synthetic probe
accggcaccatgggagtnggttgcaccagaagccggt

SEQ ID NO: 25 - DNA, 84 bp, Bacillus subtilis
acaccacgagagtttgtaacacccgaagtcggtgaggtaaccttttaggagccagccgccgaaggtgggacagatgattggggt

SEQ ID NO: 26 - DNA, 84 bp, Bacillus anthracis
acaccacgagagtttgtaacacccgaagtcggtggggtaacctttttggagccagccgcctaaggtgggacagatgattggggt

SEQ ID NO: 27 - DNA, 83 bp, Bacillus sphaericus
acaccacgagagtttgtaacacccgaagtcggtggggtaaccttttggagccagccgccgaaggtgggatagatgactggggt

SEQ ID NO: 28 - DNA, 83 bp, Bacillus thermoamylovorans
acaccacgagagtttgtaacacccgaagtcggtgaggtaaccgtaaggagccagccgccgaaggtgggacagatgattggggt

SEQ ID NO: 29 - DNA, 82 bp, Bacillus subtilis
acaccacgagagtttgtaacacccgaagtcggtgaggtaaccttttaggagccagccgccgaaggtgggacagatgattggg

SEQ ID NO: 30 - DNA, 82 bp, Bacillus anthracis
acaccacgagagtttgtaacacccgaagtcggtggggtaacctttttggagccagccgcctaaggtgggacagatgattggg

SEQ ID NO: 31 - DNA, 81 bp, Bacillus sphaericus
acaccacgagagtttgtaacacccgaagtcggtggggtaaccttttggagccagccgccgaaggtgggatagatgactggg

SEQ ID NO: 32 - DNA, 81 bp, Bacillus thermoamylovorans
acaccacgagagtttgtaacacccgaagtcggtgaggtaaccgtaaggagccagccgccgaaggtgggacagatgattggg

SEQ ID NO: 33 - DNA, 81 bp, Staphylococcus epidermidis
acaccacgagagtttgtaacacccgaagccggtggagtaaccatttggagctagccgtcgaaggtgggacaaatgattggg

SEQ ID NO: 34 - DNA, 84 bp, Streptomyces griseus
acgtcacgaaagtcggtaacacccgaagccggtggcccaaccccttgtgggagggagctgtcgaaggtgggactggcgattggg

SEQ ID NO: 35 - DNA, 82 bp, Mycobacterium lacus
acgtcatgaaagtcggtaacacccgaagccagtggcctaaccctttgggagggagctgtcgaaggtgggatcggcgattggg

SEQ ID NO: 36 - DNA, 84 bp, Nocardia cyriacigeorgica
acgtcatgaaagtcggtaacacccgaagccggtggcctaaccccttgtgggagggagccgtcgaaggtgggatcggcgattggg

SEQ ID NO: 37 - DNA, 23 bp, Artificial Sequence
  Description of Artificial Sequence Primer
gaagtcgtaacaaggtatccgta

SEQ ID NO: 38 - DNA, 124 bp, Escherichia coli
ttgtacacaccgcccgtcacaccatgggagtgggttgcaaaagaagtaggtagcttaaccttcgggagggcgcttaccactttgtgattcatgactggggtgaagtcgtaacaaggtaaccgta

SEQ ID NO: 39 - DNA, 125 bp, Bacillus subtilis
ttgtacacaccgcccgtcacaccacgagagtttgtaacacccgaagtcggtgaggtaaccttttaggagccagccgccgaaggtgggacagatgattggggtgaagtcgtaacaaggtagccgta

SEQ ID NO: 40 - DNA, 150 bp, Aspergillus oryzae
ttgtacacaccgcccgtcgctagtaccgattgaatggcttagtgaggcctcaggatctgcttagagaagggggcaactccatctcagagcggagaatttggacaaacttggtcatttagaggaactaaaagtcgtaacaaggtttccgta

SEQ ID NO: 41 - DNA, 152 bp, Spinacia oleracea
ttgtacacaccgcccgtcgctcctaccgattgaatgatccggtgaagtgttcggattgcgtcgacgacggtggttcgccgctgtcgacgtcgtgagaagttcattgaaccttatcatttagaggaaggagaagtcgtaacaaggttaccgta

(19) United States
(12) Patent Application Publication
(10) Pub. No.: US 2004/0176584 A1
(43) Pub. Date: Sep. 9, 2004

(54) Methods for detecting, enumerating, quantifying, classifying and/or identifying total organismal DNA in a sample

(75) Inventor: Kathy Terlesky, Charlottesville, VA (US)

Correspondence Address: MILLEN, WHITE, ZELANO & BRANIGAN, P.C., 2200 CLARENDON BLVD., SUITE 1400, ARLINGTON, VA, 22201, US

(73) Assignee: Veridian Sysytems Division, Arlington, VA

(21) Appl. No.: 10/360,935

(22) Filed: Feb. 10, 2003

(51) Int. Cl. C12Q 1/68; C07H 21/04

(52) U.S. Cl. 536/24.3; 435/6

(57) ABSTRACT

A universal primer pair (SEQ. ID 1 and 2) is used to amplify total organismal DNA in a sample. Probes from any or all three taxonomic domains may be used to further classify the amplified DNA by organelle (e.g., chloroplast) or by taxonomic DNA levels (i.e., kingdom, phylum, class, order, or family) in relation to total DNA. The universal primer pair amplifies regions of the 16s (eukaryotic 18s) ribosomal DNA gene by hybridizing to regions highly conserved among all organismal DNA. Less conserved regions within the amplicon produced by the universal primers are targeted by probes, so that a general taxonomic breakdown of the DNA present can be determined. The primers and probes of the present invention are conveniently used with real-time PCR or similar or related technology to detect and enumerate any organismal DNA from the three major taxonomic groups (bacteria, archaea and eucarya) and enable improved methods of environmental surveillance and quick identification of unknown biological material.

The invention was made with government support under contract #DAAD05-00-C-7113. The government has certain rights in this invention.

### SUMMARY OF THE INVENTION

The present invention relates generally to methods for detecting, enumerating, quantifying, classifying and/or identifying total organismal DNA in a sample. More particularly, the present invention provides a method for determining total DNA content in a sample by detecting the presence of nucleotide sequences associated with all or part of 16s(18s) rDNA or homologues, functional equivalents or derivatives thereof, e.g., as might be naturally occurring in various organisms or as might occur as a result of environmental effects such as natural or human caused mutations, etc. The nucleotide sequences of the present invention may be used as indicators of any DNA and, hence, represent universal target sequences which are indicative of total DNA content in a sample. The universal target sequence may also be used to capture DNA which may be subsequently analyzed, e.g., by sequence analysis or genetic probe technology. The universal target sequence is useful in designing universal primers and probes to amplify any genomic sequence, as a means to detect and enumerate total DNA and to further identify DNA in a sample at a taxon specific level. Furthermore, the development of a universal primer-probe set permits the rapid and accurate determination of total organismal DNA load without necessitating the development of multiple specific primers for particular species. Such uses enable improved methods of environmental surveillance, environmental protection, bioremediation, diagnostics, industrial microbiology and the like. The present invention further relates to the universal target sequence in isolated form and/or primers or probes capable of hybridizing to same and devices for the detection of total DNA in a sample, etc.

Various other features and attendant advantages of the present invention will be more fully appreciated as the same becomes better understood when considered in conjunction with the accompanying drawings, in which like reference characters designate the same or similar parts throughout the several views, and wherein:

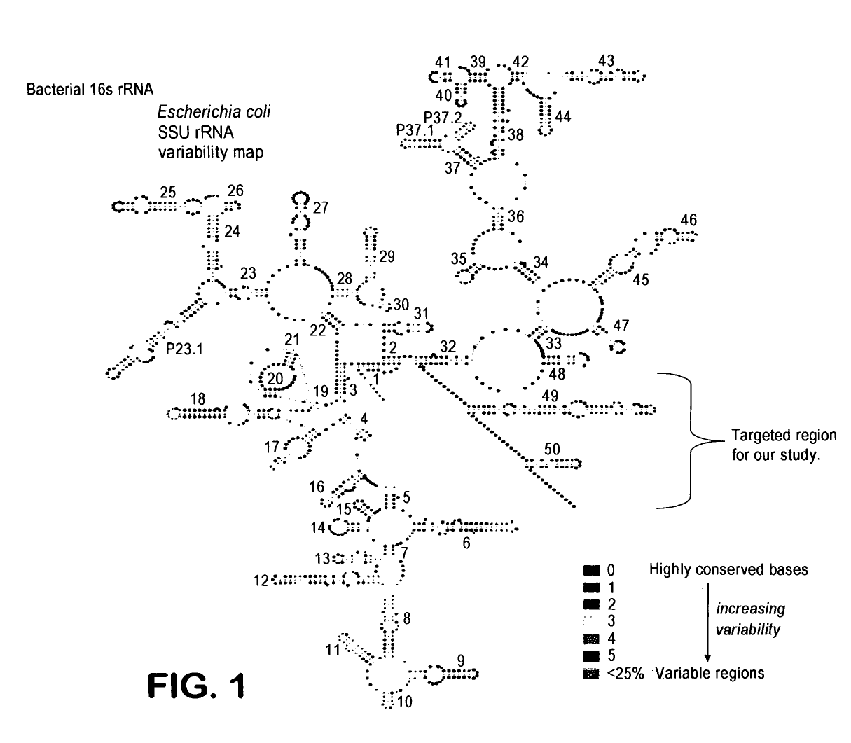

FIG. 1 shows a map of E. coli 16s rRNA and the target region of study.

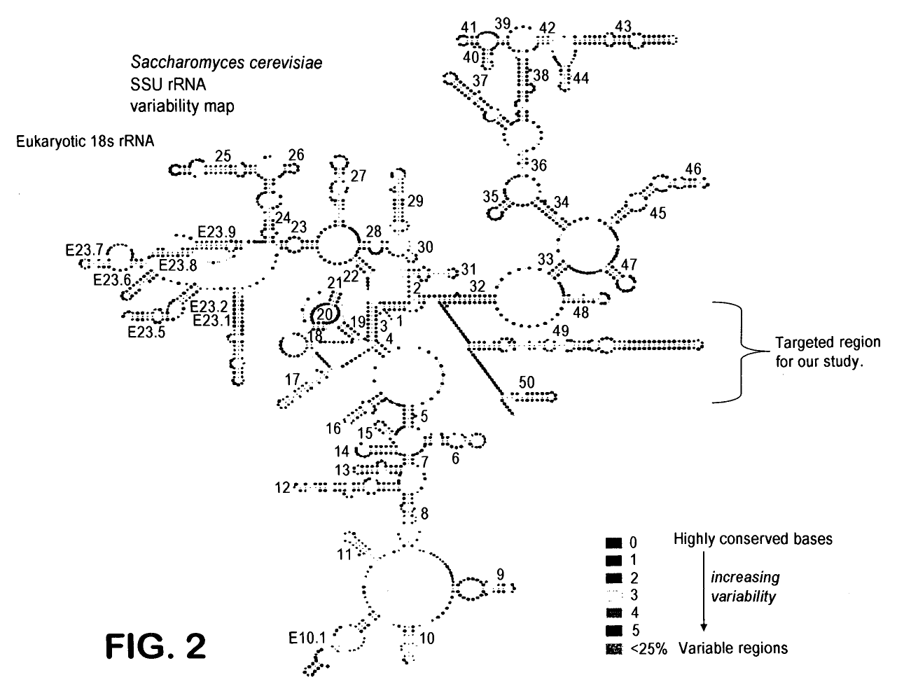

FIG. 2 SHOWS a map of Saccharomyces cerevisiae 18s rRNA and the target region of study.

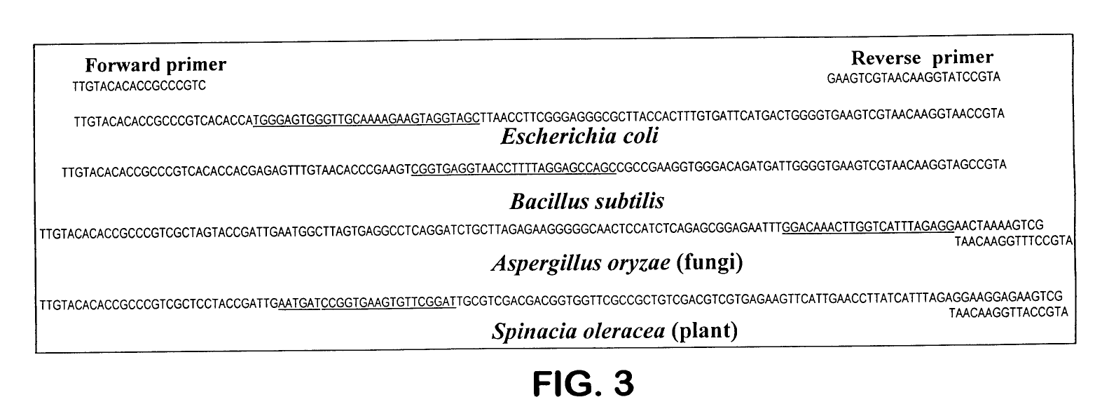

FIG. 3 SHOWS the DNA sequence alignment of conserved sequences used as primers in the methods and compositions of the invention.

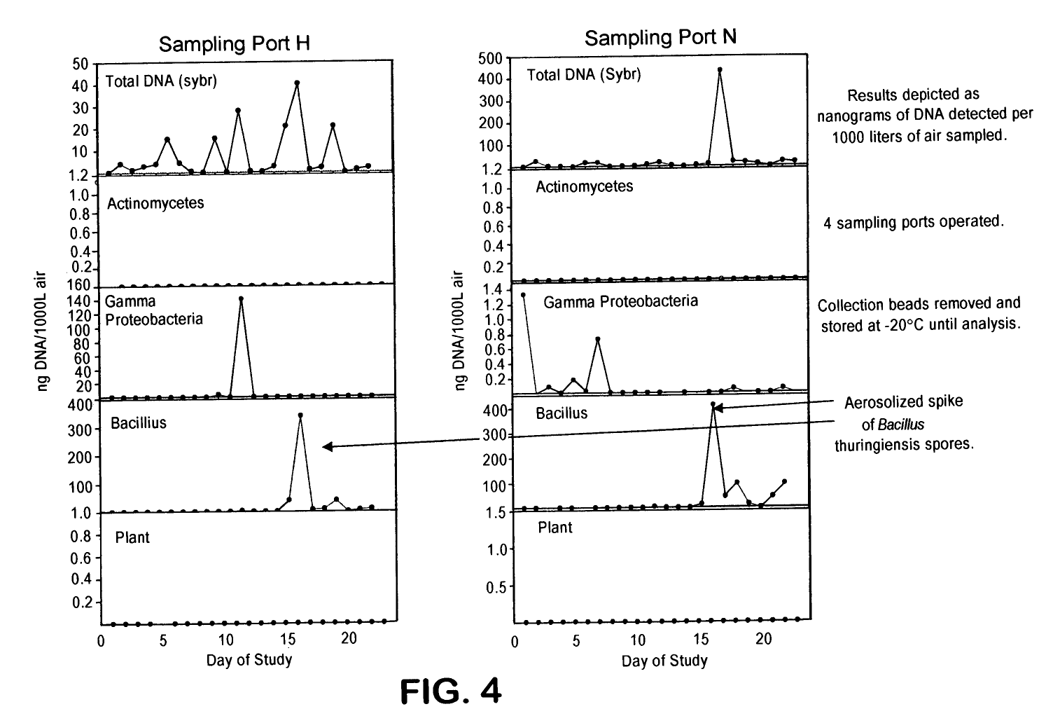

FIG. 4 depicts results of air sampling as nanograms of DNA detected per 1000 liters of air sampled. Aerosolized spikes of Bacillus thuringiensis spores can be seen at day 15.

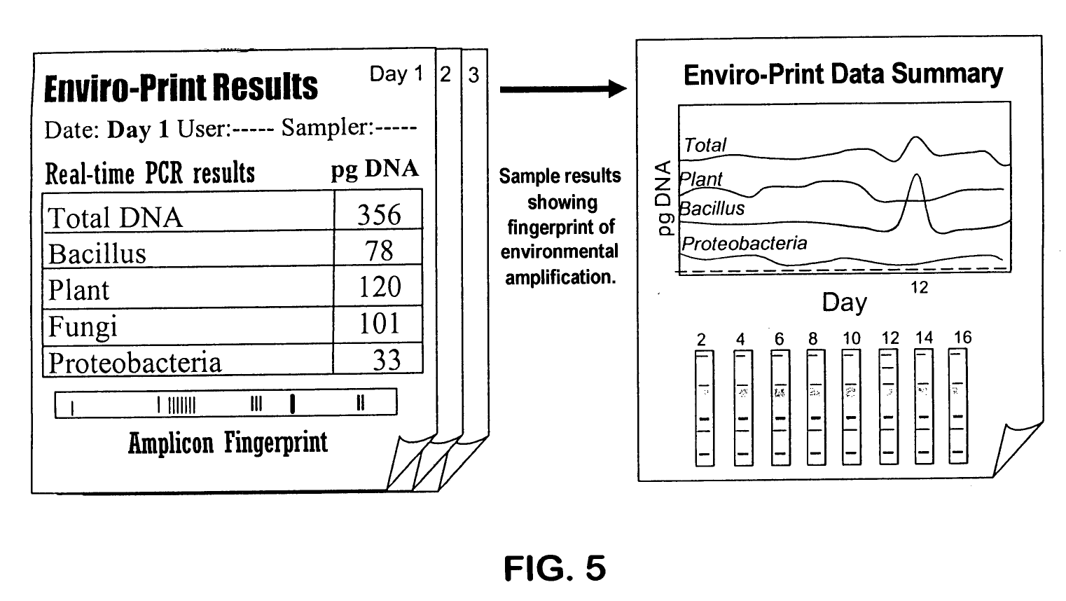

FIG. 5 shows a sample amplicon fingerprint on day one and a graphical representation of an environmental fingerprint over time. Values for total DNA, plant, bacillus and proteobacteria were recorded.

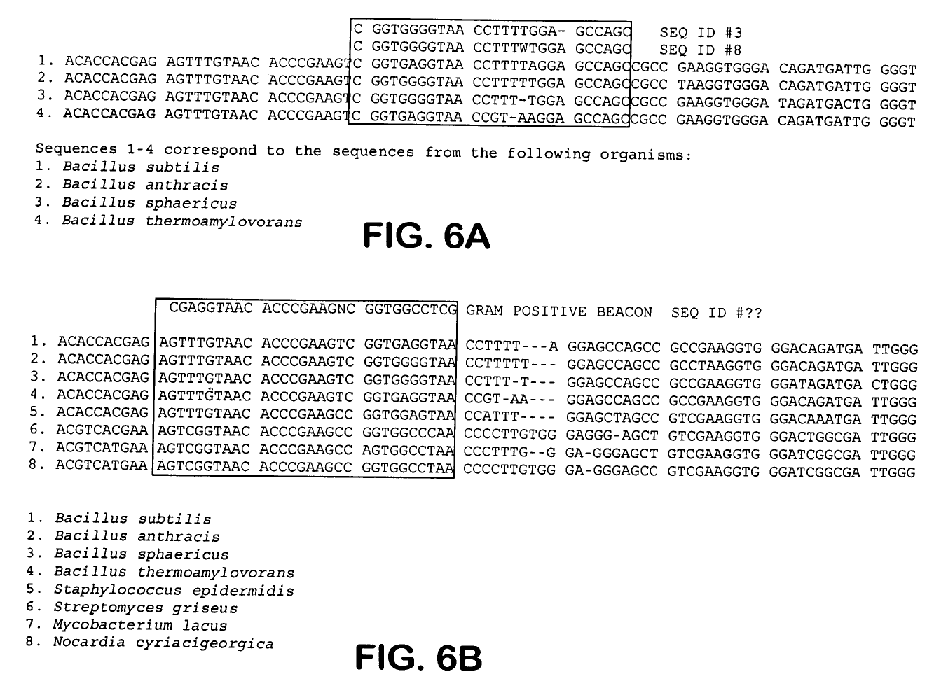

FIGS. 6A and 6B show sample sequence alignments for a class specific probe (Bacilli class) and a division-specific probe (Division Formicutes). Only the region between the conserved universal forward (SEQ ID #1) and universal reverse (SEQ ID #2) primers is shown. FIG. 6A shows the sequence alignments between Bacilli class-targeted probes and the target amplification regions from four different Bacillus species. FIG. 6B shows the sequence alignments between a gram-positive beacon probe and the target amplification region of different gram-positive bacteria.

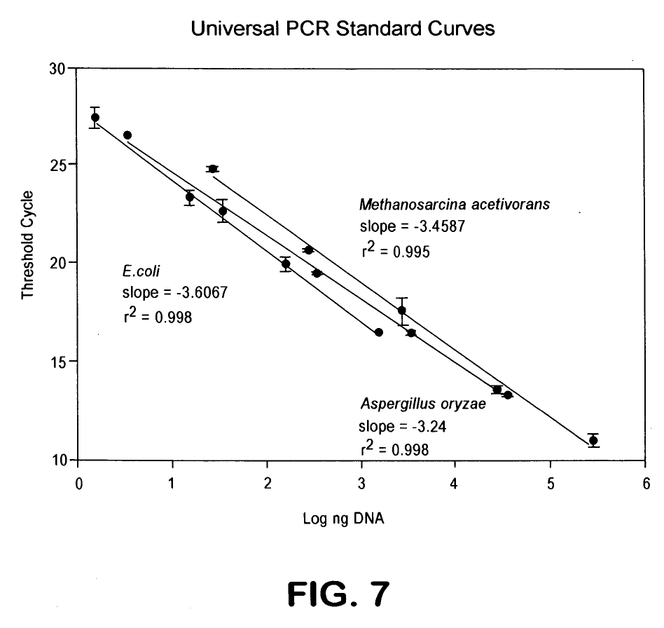

FIG. 7 shows the PCR standard curve generated using DNA from 3 separate domains of organisms (Archaea (Methanosarcina acetivorans), Bacteria (E. coli) and Eukarya (Aspergillus oryzae)).

This invention provides new methods of obtaining genomic fingerprint information directly from all organismal DNA in any environmental setting, typically where such organisms have 16s or 18s DNA, and to primers and probes used in such methods. The present invention takes advantage of the fact among others that certain coding sequences are highly conserved in all organisms. In addition to the 16s(18s) regions, other regions that are central to the major cellular processes conserved among all organisms may be used, such as, for example, the tRNA genes or the 23s and 5s tRNA genes. The tRNA gene is short and amplified fragments are small making it an ideal target region for the PCR approach. By properly choosing PCR primers from among these conserved sequences, one set of PCR primers (or a set of degenerate primers) can be used for the amplification of an unknown DNA sample (with several possible and different genomic origins) for the purpose of revealing total DNA values. The system can be optimized for use in real-time PCR methods by selecting the conserved primer regions within a 500 bp range.

The invention provides a method of classifying total DNA in real-time from a population of organisms in a biological sample, comprising obtaining genetic material from the sample; contacting the genetic material with a universal primer pair corresponding to a pair of conserved regions in the genomes of the population of organisms, wherein the first primer hybridizes upstream and the second primer hybridizes downstream of a target sequence in the genetic material in the sample, wherein the target sequence is less conserved than the primer binding sequences and is characteristic of a particular organism; and/or amplifying the target sequence.

The invention further relates to a broad-range panel of organelle and taxon-specific 16s (18s) based rDNA probes to identify organisms at the organelle, kingdom, phylum, class, order, or family specificity level. The probes are useful either individually or in a panel for identifying and detecting higher taxa directly from samples and provide insight into the ecological diversity of the community of organisms present. Of course other methods of specifying the organism can be used, e.g., restriction analysis, etc.

For example, the invention further relates to primers, comprising, consisting essentially of, or consisting of, a forward primer of SEQ ID NO. 1 or a sequence having at least 80-85%, 90-95% or 97-99% etc identity to SEQ ID NO. 1 or a sequence capable of hybridizing to SEQ ID NO. 1 under low stringency conditions

and a reverse primer of SEQ ID NO. 2 or a sequence having at least 80-85%, 90-95% or 97-99% etc identity to SEQ ID NO. 2 or a sequence capable of hybridizing to SEQ ID NO. 2 under low stringency conditions.

Furthermore, the invention relates to a primer pair, comprising, consisting essentially of, or consisting of, the amino acid sequences of SEQ ID NO: 1 and 2 or or sequences having at least 80-85%, 90-95% or 97-99% etc identity to SEQ ID NO. 1 and 2 or sequences capable of hybridizing to SEQ ID NO. 1 and 2 under low stringency conditions.

In addition, the invention further relates to probes, comprising, consisting essentially of, or consisting of, the amino acid sequence of SEQ ID NO: 3, 4, 5, 6, 7, 8, 9, 10, 11, 12, 13, 14, 15, 16, 17, 18, 19, 21, 22, 23, 24 or a fragment or variant of SEQ ID NO: 3, 4, 5, 6, 7, 8, 9, 10, 11, 12, 13, 14, 15, 16, 17, 18, 19, 21, 22, 23, or 24. The probe may comprise, e.g., at least about 7, 10, 12 or 14 etc. contiguous amino acids of SEQ ID NO: 3, 4, 5, 6, 7, 8, 9, 10, 11, 12, 13, 14, 15, 16, 17, 18, 19, 21, 22, 23, 24; and/or may have a sequence identity of, e.g., at least about 80-85%, 90-95% or 97-99% etc. to SEQ ID NO: 3, 4, 5, 6, 7, 8, 9, 10, 11, 12, 13, 14, 15, 16, 17, 18, 19, 21, 22, 23, 24.

To aid in understanding the invention, several terms are defined below and apply in singular or plural form.

The term “organismal DNA” as used herein refers to DNA derived from any of the three domain designations of Woese. Archaea are sometimes called Archeobacteria, but are not true bacteria. Bacteria include prokaryotes (true bacteria). Eucarya refers to all organisms with eukaryotic cells. Eucarya include four of the five Whittaker's kingdoms: protists, fungi, plants and animals. The term encompasses substantially all DNA derived from organisms (i.e., up to and including 100% but not necessarily 100%).

The term “taxonomy or taxonomic” refers to the science of biological classification. In a broader sense it consists of three separate but interrelated parts: classification, nomenclature, and identification. In the Whittaker taxonomic system there are five kingdoms of living organisms and classification of organisms has traditionally begun with the kingdom. Recently, advances in DNA and chemical techniques have shown that an even more inclusive classification scheme could be devised. In the new classification scheme, domain has been placed above kingdom in taxonomic ranking. This level of ranking was created in light of the research of Carl Woese. In the Three Domain taxonomy of life, kingdom is the second of the eight ranks. Although there may also be a subkingdom/domain classification between ‘kingdom’ and ‘phylum’, it is not always used in biological identification. The major designations, listed in terms of increasing specificity, include domain, kingdom, phylum, class, order, family, genus and species. To further facilitate grouping similar or closely related groups, these taxa may be further divided with up to three named intermediate-level taxa, as required (i.e., class-major division, subclass-optional, infraclass-optional, superorder-optional, order-major division).

The term “oligonucleotide” refers to a molecule comprised of two or usually more deoxyribonucleotides or ribonucleotides, such as primers, probes, nucleic acid fragments to be detected, and nucleic acid controls. The exact size of an oligonucleotide depends on many factors and the ultimate function or use of the oligonucleotide. It can include 5,10,15,20,25,30,40,50,etc. nucleotides or more or less. Oligonucleotides can be prepared by any suitable method, including, for example, cloning and restriction of appropriate sequences and direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Lett. 22:1859-1862; and the solid support method of U.S. Pat. No. 4,458,066.

The term “primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different deoxyribonucleoside triphosphates and an agent for polymerization (i.e., DNA polymerase or reverse transcriptase) in an appropriate buffer and at a suitable temperature. A primer is preferably a single-stranded oligodeoxyribonucleotide. The appropriate length of a primer depends on the intended use of the primer and/or the incorporation of modified nucleotide residues to tailor the melting temperature characteristics, but typically ranges from 12 to 25 nucleotides. Shorter primer molecules generally require cooler temperatures to form sufficiently stable hybrid complexes with the template. A primer need not reflect the exact sequence of the template but must be sufficiently complementary to hybridize with a template and serve to initiate DNA synthesis.

The term “probe” refers to an oligonucleotide or polynucleotide that is capable of hybridizing to another nucleic acid of interest. A nucleic acid probe may occur naturally as in a purified restriction digest or be produced synthetically, recombinantly or by PCR amplification. As used herein, the term “probe” refers to the oligonucleotide or polynucleotide used in a method of the present invention. Of course the percentage of similarity would depend on the location of any mismatches. For example, a TAQMAN probe of 27 base pairs could tolerate up to 6 mismatches if the mismatches were either in the middle or near the 3′ end. In general, mismatches are less tolerated at the 5′ end since the TAQ polymerase likes a strong 5′ annealing end to perform the exonucleoytic activity. For BEACON probes, the mismatches can often be tolerated at the ends or internally since it is based on a hybridization reaction. In addition to traditional oligonucleotide probes, peptide nucleic acid probes may also be utilized. PNA (peptide nucleic acids) are DNA mimics where the sugar phosphate backbone of DNA is replaced by a neutral polyamide backbone formed by repetitive units of N-(2-aminoethyl) glycine (Egholm M: Nature Oct. 7, 1993; 365(6446): 566-8). Nucleotide bases are attached to each unit allowing the PNA's to hybridize to complementary nucleic acid sequences. PNA's have been used for nucleic acid detection by incorporating a dye (e.g., thiazole orange) into the PNA probe. The tethered dye allows the probes to effectively light up upon hybridization in a homogenous solution and thus these probes are often referred to as “light up probes”. They are fully conventional (See for example, Svanvik N: Anal Biochem May 15, 2000;281(1):26-35 or Isacsson J :Mol Cell Probes October 2000;14(5):321-8).

For the purposes of the present invention, when a probe or a primer is identified by its sequence, such probe or primer shall be taken to include the complementary sequence.

This invention may be applied also to the ribosomal RNA molecules present in cells. For example, by isolating total RNA or ribosomal RNA from a sample and performing reverse transcription, the rRNA or other RNA of interest would be copied into cDNA. The cDNA would then serve as the template for universal PCR amplification. The enzyme, reverse transcriptase, copies a strand of mRNA or rRNA into DNA using a DNA oligonucleotide primer. The RNA is then degraded and the copied DNA fragment could be used directly in PCR amplification. Alternatively, it is also possible to amplify the RNA per se using appropriate enzymes and RNA based primers corresponding to those described herein.

As used herein, the terms “complementary” or “complementarity” are used in reference to nucleic acids (i.e., a sequence of nucleotides) related by the well-known base-pairing rules that A pairs with T and C pairs with G. For example, the sequence 5′-A-G-T-3′, is complementary to the sequence 3′-T-C-A-5′.

Complementarity can be “partial,” in which only some of the nucleic acid bases are matched according to the base pairing rules. On the other hand, there may be “complete” or “total” complementarity between the nucleic acid strands when all of the bases are matched according to base pairing rules. The degree of complementarity between nucleic acid strands often has significant effects on the efficiency and strength of hybridization between nucleic acid strands as known well in the art. The term “substantially complementary” refers to any probe that can hybridize to either or both strands of the target nucleic acid sequence under stringent conditions as described below.

As used herein, the term “hybridization” is used in reference to the pairing of complementary nucleic acid strands. Hybridization and the strength of hybridization (i.e., the strength of the association between nucleic acid strands) is impacted by many factors well known in the art including the degree of complementarity between the nucleic acids, stringency of the conditions involved affected by such conditions as the concentration of salts, the Tₘ (melting temperature) of the formed hybrid, the presence of other components, the molarity of the hybridizing strands and the G:C content of the nucleic acid strands, etc.

As used herein, the term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required when it is desired that nucleic acids which are not completely complementary to one another be hybridized or annealed together. One skilled in the art knows well that numerous equivalent conditions can be employed to comprise any degree of stringency. In a preferred embodiment, probe hybridization is performed at high stringency. Reference herein to low stringency includes and encompasses for example, less then 16% v/v formamide and at least about l M to at least about 2 M salt for hybridization, and at least about 1 M to at least about 2 M salt for washing conditions. Generally, low stringency is at least from about 25-30° C. to about 42° C. The temperature may be altered and higher temperatures used to replace formamide (if used) and/or to give alternative stringency conditions. Alternative stringency conditions may be applied where necessary, such as medium stringency, which includes and encompasses from at least about 16% v/v to at least about 30% v/v formamide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. Formamide is optional in these hybridization conditions. Accordingly, particularly preferred levels of hybridization stringency are defined as follows.

Low stringency is 6×(SSC) buffer, 0.1% w/v sodium dodecyl sulphate (SDS) at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

With respect to stringency conditions in PCR generally, low stringency is at least from about 30-45° C. with 1-10 mM MgCl₂ and/or 15 to 30° C. below the Tm for the primers. Medium stringency includes and encompasses from about 45-60° C. with 1-10 mM MgCl₂ and/or 0-15° C. below the Tm for the primers. High stringency includes and encompasses from about 60-80° C. with 1-10 mM MgCl₂ and/or 0 to 15° C. above the Tm for the primers.

With respect to preferred probe stringency conditions in real-time PCR, low stringency is at least from about 30-45° C. with 1-10 mM MgCl₂. Medium stringency includes and encompasses from about 45-60° C. with 1-10 mM MgCl₂ and high stringency includes and encompasses from about 60-80° C. with 1-10 mM MgCl₂.

The terms “fragment” or “variant,” when referring to an oligonucleotide of the invention mean an oligonucleotide which retains substantially at least one of the functions or activities of the oligonucleotides of SEQ ID NOs 1-24. Fragments or variants of the oligonucleotides, e.g. of SEQ ID NOs 1-24, have sufficient similarity or identity to those oligonucleotides so that at least one activity of the oligonucleotides is retained. Oligonucleotide fragments of the invention may be of any size that is compatible with the invention. The term “fragment” further refers to a sequence that is a subset of a larger sequence (i.e., a continuous or unbroken sequence of residues within a larger sequence) and is of a length to be specific (i.e., uniquely related to the latter).

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100 [1−(C/R))

wherein C is the number of differences between the Reference Sequence and the Compared Sequence over the length of alignment between the Reference Sequence and the Compared Sequence wherein (i) each base in the Reference Sequence that does not have a corresponding aligned base in the Compared Sequence and (ii) each gap in the Reference Sequence and (iii) each aligned base in the Reference Sequence that is different from an aligned base in the Compared Sequence, constitutes a difference; and R is the number of bases in the Reference Sequence over the length of the alignment with the Compared Sequence with any gap created in the Reference Sequence also being counted as a base or amino acid.

The techniques for determining oligonucleotide sequence “similarity” and/or “identity” are well known in the art. The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLASST) can be used.

In the examples and disclosed embodiments of the invention, specific sequence primers and probes are provided. It will be apparent to those of skill in the art that, provided with those embodiments, specific sequence primers and probes can be modified conventionally by, for example, the addition of nucleotides to either the 5′ or 3 ′ ends, which nucleotides are complementary to the target sequence or are uncomplementary to the target sequence. Often they are modified bases capable of additional H-bonding, but will amplify normally. Modification of the primer 5′ ends for post PCR manipulation or other uses is fully conventional. (See Scharf S J; Science Sep. 5, 1986; 233(4768):1076-8.) In addition, modification of nucleotides to adjust the Tm of primers is fully conventional. (See “Nucleic Acid Hybridization and Unconventional Bases” at pages 69-83 and Igor V; Biochemistry 35:1170-1176 (1996).)

So long as primer compositions serve as a point of initiation for extension on the target sequences, and the primers and probes comprise at least about 7 consecutive nucleotides contained within those exemplified embodiments, such compositions are within the scope of the invention. In general, for primers and TAQMAN probes, a minimum stretch of 7 nucleotides at each end is sufficient. The primers and probes can typically tolerate 1-3 mismatches in the internal regions of the primers and TAQMAN probes, generally, not more than 3 mismatches total. Beacon probes, can often tolerate 3-5 mismatched nucleotides at each end and then generally have at least the 7 consecutive nucleotides following a mismatch. As with the TAQMAN probes, the internal sequence of the beacon probes often can tolerate 1-3 mismatches and still remain functional.

The term “primer pair” refers to a matched pair of primers having a forward primer that hybridizes to a region upstream of a target sequence and a reverse primer that hybridizes to a region downstream of a target sequence. A primer can be labeled, if desired, by incorporating a label detectable by spectroscopic, photochemical, biochemical, immunochemical, or chemical means. For example, useful labels include ³²P, fluorescent dyes, electron-dense reagents, enzymes (as commonly used in ELISAs), biotin, or haptens and proteins for which antisera or monoclonal antibodies are available.

The term “sequence-specific oligonucleotide” refers to oligonucleotides that have a sequence, called a “hybridizing or annealing region”, complementary to the sequence to be detected, which, under “sequence specific, stringent hybridization or annealing conditions”, will hybridize or anneal only to that exactly complementary target sequence. Relaxing the stringency of the hybridization conditions or altering the annealing temperatures will allow sequence mismatches to be tolerated; the degree of mismatch tolerated can be controlled by suitable adjustment of the hybridization conditions. All of this is conventional.

The term “target region” refers to a region of a nucleic acid to be analyzed.

The term “thermostable polymerase enzyme” refers to an enzyme that is relatively stable to heat and catalyzes the polymerization of nucleoside triphosphates to form primer extension products that are complementary to one of the nucleic acid strands of the target sequence. The enzyme initiates synthesis at the 3′-end of the primer and proceeds in the direction toward the 5′-end of the template until synthesis terminates. A purified thermostable polymerase enzyme is described more fully in U.S. Pat. No. 4,889,818, incorporated herein by reference, and is commercially available from Perkin Elmer (Norwalk, Conn.). These enzymes and there use are fully conventional.

The term “Real-time PCR” refers to fully conventional systems such as the TaqMan (Registered trade mark) system developed by AppliedBiosystems which relies on the release and detection of a fluorogenic probe during each round of DNA amplification. It allows for the rapid detection and quantification of DNA without the need for post-PCR processing such as gel electrophoresis and radioactive hybridization. In addition, the built-in 96 well format greatly increases the number of samples that can be simultaneously analyzed. The method uses the 5′ exonuclease activity of a Taq polymerase during primer extension to cleave a dual-labelled, fluorogenic probe hybridized to the target DNA between the PCR primers. Prior to cleavage, a reporter dye, such as 6-carboxyfluorescein (6-FAM) at the 5′ end of the probe is quenched by 6-carboxy-tetramethylrhodaniine (TAMRA) through fluorescent resonance energy transfer. Following digestion, FAM is released. The resulting fluorescence is continuously measured in real-time at around 518 nm during the log phase of product accumulation and is proportional to the number of copies of the target sequence.

Alternative real-time PCR amplification and detection systems utilize FRET probes or Beacon probes (hybridization probes) that do not rely on the exonuclease activity of the TAQ for signal generation.

There are numerous apparatus available for collecting sample DNA material from an environment. For example, the Anderson sampler collects airborne particles by impingement onto culture plate surfaces. The AGI-30 collects airborne particles by impingement into a liquid medium facilitating both culture dependent or independent methods of post-collection analysis. There are numerous other suitable collection devices such as, for example, those disclosed in U.S. 2002/0019060, U.S. 2002/0062702, U.S. Pat. No. 6,337,213, and U.S. Pat. No. 5,766,958. The preferred method of sample collection is an air sampling system based on the collection of airborne particles. A packed bed of glass beads is coated with a sticky aerogel coating that facilitates collection of airborne particles as air is drawn into the collection ports and over the beads. Preferred aerogel coatings for example, are those disclosed in U.S. 60/375,790 and U.S. 60/375,905 entitled Glycerol-Doped Aerogel Coatings as Biological Capture Media. Bead beating to achieve cell lysis and liberate DNA is one way to extract the total DNA material collected on the beads. Once extracted, the DNA is subjected to post collection analysis using the universal primers and probes of the invention. Many other collection and retrieval methods are fully conventional and can be used in conjunction with this invention.

The design and selection of primers is an important aspect of any project involving PCR. One aspect of the invention focuses on a discovered pair of universal primers derived from regions of 16s rDNA gene (and 18s rDNA) sequences that are highly conserved among all organismal DNA (e.g. bacteria, archaea and eucarya). Generally, primers should be 12-25 nucleotides long with closely matched melting temperatures (less than 5° C. difference) and have at least a 5 base match at the 3′end. It is preferable that mismatches are at the 5′ end. Modification of stringency conditions and temperatures are well known in the art. Conventional principles are applicable, of course. The base content of an oligonucleotide affects the denaturation temperature, the stringency and specificity of primer or probe binding increases with increasing temperature, etc.

The primer pairs of the invention function efficiently in the amplification of a sequence of the 16s (eukaryotic 1 8s) rDNA gene from organisms across all three of the major taxonomic domains (e.g., bacteria, archaea and eucarya). Furthermore, the amplification conditions and efficiency for these primers are fairly uniform across all species so that nearly all-organismal DNA is detectable using a single test. Table 1 shows the preferred hybridizing sequences of the primers of the present invention.

TABLE I

Sequence

| | |
|---|---|
| SEQ ID NO: 1 | |
| Forward Primer (5′ to 3′): | TTGTACACACCGCCCGTC |
| SEQ ID NO: 2 | |
| Reverse Primer (5′ to 3′): | TACGGNNACCTTGTTACGACTT, |

where “N” may be inosine or any of the other 4 nucleotide bases

Together, these primers specify the synthesis of products ranging from approximately 90 to 200 base pairs in length; the exact size is species dependent. The primers may also be modified with flanking sequences on the 5′ end that facilitate post-amplification manipulation and analysis and/or with any other compatible modification.

The initial screening for the presence of specific organismal DNA, from the DNA amplified using the inventive primer pair, can be accomplished with five taxon specific probes and a chloroplast probe that may be used simultaneously as a mixture or independently. The selection and number of probes used in a panel will be partly dependent on the environment of the sample tested, and can be optimized readily. Table 2 shows certain exemplary and non-limiting probes which can be used to identify certain organisms.

TABLE 2

Sequence Listing
Probe Type
Hybridizing Sequence
SEQ ID NO: 3
Bacillus
5′ CGG TGG GGT AAC CTT TTG GAG CCA GC 3′
SEQ ID NO: 4
Actinomycete
5′ CGG TGG CCC AAC CCC TTG TGG GA 3′
SEQ ID NO: 5
Gamma proteobacterial
5′ TGG GAG TGG GTT GCA AAA GAA GTA GGT AGC 3′
SEQ ID NO: 6
Fungal
5′ AAG TCG TAA CAA GGT TTC CGT AGG TGA ACC 3′
SEQ ID NO: 7
Plant
5′ CGA AGT CGT TAC CTT AAC CGC AAG 3′

Other non-limiting specific probes, which can be used in an identification step are shown in Table 3.

TABLE 3

Sequence Listing
Probe Type
Hybridizing Sequence
SEQ ID NO: 8
Bacillus2
5′ CGG TGG GGT AAC CTT TWT GGA GCC AGC 3′
SEQ ID NO: 9
Plant
5′ TCC GGT GAA GTG TTC GGA TC 3′
SEQ ID NO: 10
Gram-positive bacterial
5′ CGTACGTAACACCCGAAGNGGGTGGCGTACG
SEQ ID NO: 11
Gram positive bacterial (beacon probe)
5′ ACA CCA CGA GAG TTN GTA ACA CCC GAA GT 3′
SEQ ID NO: 12
Alpha proteobacterial
5′ ACC CGA AGG CGC TGC GCT AA 3′
SEQ ID NO: 13
Fungal
5′ CAA ACT TGG TCA TTT AGA GGA AGT 3′
SEQ ID NO: 14
Pezizomycotina
5′ TG AGG CCT TCG GAC TGG CTC 3′
SEQ ID NO: 15
Enterobacteria
5′ TTA ACC TTC GGG AGG GCG CTT AC ACT TT 3′
SEQ ID NO: 16
Green algae
5′ CGA TTG GGT GTG CTG GTG AAG TGT T 3′
SEQ ID NO: 17
Actinobacterial (beacon probe 1)
5′ CGA GGT AAC ACC CGA AGN CGG TGG CCT CG 3′
SEQ ID NO: 18
Gram positive (beacon probe 2)
5′ CGC ACC ACG AAA GTT NGT AAC ACC CGA AGG TGC G 3′
SEQ ID NO: 19
Plant (beacon probe)
5′ ATC CGG TGA AGT GTT CGG ATC 3′
SEQ ID NO: 20
Chloroplast (beacon probe)
5′ CTT GCG AAG TCG TTA CCT TAA CCG CAA G 3′
SEQ ID NO: 21
Pezizomycotina (beacon probe)
5′ CGT GAG GCC TTC GGA CTG GCT CAC G 3′
SEQ ID NO: 22
Fungal (beacon probe)
5′ CGC AAA CTT GGT CAT TTA GAG GAA GTT TGC G 3′
SEQ ID NO: 23
Ascomycota (beacon probe)
5′ CCG GCA ACG ACC ACC CAG GGC CGG 3′
SEQ ID NO: 24
Proteobacterial (beacon probe)
5′ ACC GGC ACC ATG GGA GTN GGT TGC ACC AGA AGC CGG T 3′

Probes may also be made to the opposite strand and thus the reverse complements of the above probes are also suitable for use in the present invention.

The probes of the present invention represent all three taxonomic domains and may further be used to classify DNA by taxonomic rank and DNA levels in relation to total DNA. The taxonomic specificity of the probes of the present invention varies from kingdom level probes (most general) to family level probes (most specific). The plant probe is specific to chloroplast DNA. Table 4 shows the taxonomic or organelle specificity of several of the probes of the present invention, which are exemplary only and non-limiting.

TABLE 4

| | |
|---|---|
| TAXONOMIC NAME | RANK |

SEQ ID # 3
Bacillus
5′ CGG TGG GGT AAC CTT TTG GAG CCA GC 3′

| | |
|---|---|
| Bacilli probe | Class |

SEQ ID #4
Actinomycete
5′ CGG TGG CCC AAC CCC TTG TGG GA 3′

| | |
|---|---|
| Actinomycetales probe | Order |

SEQ ID #5
Gamma proteobacterial probe
5′ TGG GAG TGG GTT GCA AAA GAA GTA GGT AGC 3′

| | |
|---|---|
| Enterobacteriaceae | Family |

SEQ ID #6
Fungal probe
5′ AAG TCG TAA CAA GGT TTC CGT AGG TGA ACC 3′

| | |
|---|---|
| Fungal probe | Kingdom |

SEQ ID #7
Plant probe
5′ CGA AGT CGT TAC CTT AAC CGC AAG 3′

| | |
|---|---|
| Chloroplast probe | Organelle (no taxonomic rank) |

SEQ ID #8
Bacillus2
5′ CGG TGG GGT AAC CTT TWT GGA GCC AGC 3′

| | |
|---|---|
| Bacilli probe | Class |

SEQ ID #9
Plant probe
5′ TCC GGT GAA GTG TTC GGA TC 3′

| | |
|---|---|
| Magnoliophyta 18S probe | Division |

SEQ ID #11
Gram positive bacterial probe
5′ ACA CCA CGA GAG TTN GTA ACA CCC GAA GT 3′

| | |
|---|---|
| Bacilli | Class |

SEQ ID #12
Alpha proteobacterial probe
5′ ACC CGA AGG CGC TGC GCT AA 3′

| | |
|---|---|
| Alpha proteo | subdivision |

SEQ ID #13
Fungal probe
5′ CAA ACT IGG TCA TTT AGA GGA AGT A3′

| | |
|---|---|
| Fungal | Kingdom |

SEQ ID #14
Ascomycete fungal probe
5′ TG AGG CCT TCG GAC TGG CTC 3′

| | |
|---|---|
| Pezizomycotina | Subphylum |

SEQ ID #15
Gamma proteobacterial probe
5′ TTA ACC TTC GGG AGG GCG CTT ACC ACT TT 3′

| | |
|---|---|
| Enterobacteriaceae | Family |

SEQ ID #16
Green algae probe
5′ CGA TTG GGT GTG CTG GTG AAG TGT T 3′

Chlorophyta Probe Division

A preferred aspect of the present invention is the amplification of a region of the 16s (18s) rDNA gene. Although real time PCR is the preferred amplification method, amplification of target sequences in a sample may be accomplished by any known method, such as, but not limited to Polymerase Chain Reaction (PCR; described in U.S. Pat. Nos. 4,683,195, 4,683,202, 4,800,159, 4,965,188), Strand Displacement Amplification (SDA; described by G. Walker et al., Proc. Nat. Acad. Sci. USA 89, 392 (1992); G. Walker et al., Nucl. Acids Res. 20, 1691 (1992); U.S. Pat. No. 5,270,184, the disclosure of which is hereby incorporated in its entirety by reference), thermophilic Strand Displacement Amplification (tSDA; EP 0 684 315 to Frasier et al.), Self-Sustained Sequence Replication (3SR; J. C. Guatelli et al., Proc Natl. Acad. Sci. USA 87, 1874-78 (1990)), Nucleic Acid Sequence-Based Amplification (NASBA; U.S. Pat. No. 5,130,238 to Cangene), the Q.crclbar. replicase system (P. Lizardi et al., BioTechnology 6, 1197 (1988)), or transcription based amplification (D. Y. Kwoh et al., Proc. Natl. Acad. Sci. USA 86, 1173-77 (1989)), etc.

Real-time quantitative PCR is a powerful method that can be used for gene expression analysis, genotyping, pathogen detection/quantitation, mutation screening and DNA quantitation. The technology uses an instrument to detect accumulation of PCR products continuously during the PCR process, thus allowing easy and accurate quantitation in the early exponential phase of PCR.

Real-time PCR makes quantitation of DNA and RNA much more precise and reproducible because it relies on CTt (threshold cycle) values determined during the exponential phase of PCR rather than endpoint. The concept of the threshold cycle (Ct) allows for accurate and reproducible quantification using fluorescence based RT-PCR. Fluorescent values are recorded during every cycle and represent the amount of product amplified to that point in the amplification reaction. The more templates present at the beginning of the reaction, the fewer number of cycles it takes to reach a point in which the fluorescent signal is first recorded as statistically significant above background, which is the definition of the (Ct) values. This will increase the throughput, because it is no longer necessary to analyze dilutions of each sample in order to obtain accurate results. For details on conventional real-time PCR, see, e.g., Biotechniques 24:954-962. 1998 and Biotechniques 27:1116-1118. 1999

The basis for detecting DNA belonging to a specific taxon is to continuously measure PCR product accumulation using a dual-labeled flourogenic oligonucleotide probe called a TaqMan® probe, or using a hybridization probe (e.g., molecular beacon, FRET probes etc). The TAQMAN probe is composed of a short (ca. 12-35 bases) oligodeoxynucleotide that is labeled with two different flourescent dyes. On the 5′ terminus is a reporter dye and on the 3′ terminus is a quenching dye. This oligonucleotide probe sequence is homologous to an internal target sequence present in the PCR amplicon. When the probe is intact, energy transfer occurs between the two flourophors and emission from the reporter is quenched by the quencher. During the extension phase of PCR, the probe is cleaved by 5′ nuclease activity of Taq polymerase thereby releasing the reporter from the oligonucleotide-quencher and producing an increase in reporter emission intensity. Fiber optic systems connect to each well in a 96-well PCR tray format. The laser light source excites each well and a CCD camera measures the fluorescence spectrum and intensity from each well to generate real-time data during PCR amplification. Software is used to examine the fluorescence intensity of reporter and quencher dyes and calculates the increase in normalized reporter emission intensity over the course of the amplification. The results are then plotted versus time, represented by cycle number, to produce a continuous measure of PCR amplification. To provide precise quantification of initial target in each PCR reaction, the amplification plot is examined at a point during the early log phase of product accumulation. This is accomplished by assigning a fluorescence threshold above background and determining the time point at which each sample's amplification plot reaches the threshold (defined as the threshold cycle number or CT). Differences in threshold cycle number are used to quantify the relative amount of PCR target contained within each tube as described previously.

The Beacon probes are also dual-labeled probes having a fluor on the 5′ end and a non-fluorescent quencher on the 3′ end. The probe is capable of self hybridization at the ends leading to quenching of the fluor when no target in present. Upon hybridization to target, the loop of the beacon hybridizes to the target sequence removing the fluor from the general proximity of the quencher and fluorescence occurs. The probes are not cleaved during amplification by the TAQ polymerase.

An additional chemistry is also available, using SYBR® Green I dye, that can provide real-time quantitative PCR information. Assays using the SYBR® Green double-stranded DNA binding dye do not require a TaqMan® probe and provide additional experimental flexibility. The incorporation of SYBR Green I dye into a real-time PCR reaction lets the user detect any double-stranded DNA generated during PCR. This provides great flexibility because no target specific probes are required, and yet both specific and non-specific products will generate signal. The universal primer pair and the probes of the present invention may be used to quantify DNA in an unknown sample by using a series of standard curves that may be amplified simultaneously with unknown DNA. A “Total DNA” standard curve is run using SYBR green detection during PCR amplification with the universal primers and no additional probes. The standard DNA is quantified using UV-VIs absorbance spectroscopy. For PCR, it is prepared and amplified in a dilution series (10-fold over a 5-log range). Following amplification, a standard curve is prepared either manually or within the software of the Real-time Instrument in which the X axis is the DNA concentration in the standards (ng DNA or copy number) and the Y axis is the threshold cycle for each of the standards. The threshold cycle of the unknowns is plotted on a graph and the DNA concentration is derived from the quadratic equation of the standard curve line.

A similar method may be used for quantification of unknown DNA using probes. For each probe in the study, a standard DNA dilution series is run using DNA having a high target identity with the probe sequence (i.e., Bacillus DNA for the Bacillus probes, Escherichia coli DNA for the Gamma proteobacterial probes). The standard DNA may then be quantified using UV-VIs absorbance spectroscopy. The unknown DNA may be amplified at the same time in a separate well or tube of the PCR samples. Following PCR, a standard curve is constructed using the threshold cycle of the standards and the known DNA concentrations. The value of the unknown is then extrapolated from the curve.

DNA samples containing multiple 16s rDNA genes can be quantified in this manner. A SYBR-based universal PCR reaction will provide a value for the total rDNA present in the sample, and use of multiple probes will provide a delineation of the total rDNA present into different taxonomic groups.

The amplification and detection aspects of this invention are in general per se conventional, unless otherwise indicated herein.

The probes disclosed herein hybridize to specific nucleic acids encoding portions of 16s(1 8s) rDNA. In particular embodiments of the invention, the oligonucleotide probe has a sequence as given by nucleotides of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO. 17, SEQ ID NO. 18, SEQ ID NO. 19, SEQ ID NO. 20, SEQ ID NO. 21, SEQ ID NO. 22, SEQ ID NO. 23, SEQ ID NO. 24 or a sequence having about at least 80% identity thereto or a sequence capable of hybridizing to one of said sequences under low stringency conditions.

As nucleic acids do not require complete similarity to hybridize, it will be apparent to those skilled in the art that the probe sequences specifically disclosed herein may be modified so as to be substantially similar to the probe sequences disclosed herein without loss of utility as specific probes. It is well-known in the art that hybridization of similar and partially similar nucleic acid sequences may be accomplished by adjusting the hybridization conditions to increase or decrease the stringency (i.e., adjusting the hybridization temperature or salt content of the buffer).

If desired the amplicons can be further separated by a secondary analytical method such as D/TGGE. Denaturing gradient gel electrophoresis (DGGE) and temperature gradient gel electrophoresis (TGGE) are similar techniques that allow PCR products of the same length but of different sequence composition to be separated in gradient gels according to the melting behavior of the DNA. DGGE involves separation of the double-stranded amplification products in a linearly increasing gradient of formamide and urea while TGGE achieves resolution with a linearly increasing temperature gradient. The techniques are ideal for analysing PCR products amplified from 16s(18s) rDNA genes in complex communities. D/TGGE analysis generates a community fingerprint, but since individual bands can be recovered and analyzed following D/TGGE analysis, sequence information can also be obtained. This allows a thorough analysis of organismal communities on several levels ranging from the community structure of dominant populations using conserved sequence primers, to phylogenetic sequence analysis of single bands generated by individual community members. (Environmental Molecular Microbiology: Protocols and Applications ED: Editor: Paul A. Rochelle Chap. 13)

The probes of the invention can be used to determine if nucleic acid sequences are present in a sample by determining if the probes bind to the sequences present in the sample. Suitable assay methods for purposes of the present invention to detect hybrids formed between probes and nucleic acid sequences in a sample are known in the art. For example, the detection can be accomplished in a neat sample using a fluorescent spectrophotometer, or in a dot blot format, or on a microarray chip, etc.

A large number of probe sequences can be deposited onto the surface of a microarray substrate. The identity of the target sequence is defined by its specific hybridization to a probe or probes on the chip. The main advantage of this method is that it can survey a large number of probes with relative ease. Accordingly, in one embodiment, oligonucleotides probes are immobilized to a solid support at defined locations (i.e., known positions). This immobilized microarray is sometimes also referred to as a “biochip”. The solid support can be, for example, a nylon (polyamide) membrane, glass slide, silicon chip, polymer, plastic, ceramics, metal, optical fiber or other material. The solid support can also be coated (e.g., with gold or silver) to facilitate attachment of the oligonucleotides to the surface of the solid support. Any of a variety of methods known in the art may be used to immobilize oligonucleotides to a solid support. A commonly used method consists of the non-covalent coating of the solid support with avidin or streptavidin and the immobilization of biotinylated oligonucleotide probes. The oligonucleotides can also be attached directly to the solid supports by epoxide/amine coupling chemistry. See Eggers et aL Advances in DNA Sequencing Technology, SPIE conference proceedings (1993).

Alternatively, it may be desirable to use a detection method having a plurality of probe hybridization sites or wells. For example, a solid support such as a microtiter plate is particularly useful in large scale applications of the present methods. U.S. Pat. No. 5,232,829, incorporated herein by reference, describes a method for hybridization/capture of PCR amplified DNA on solid supports. In one embodiment of those methods the amplified target DNA is labeled (e.g., with biotin) during amplification in the PCR reaction. The labeled DNA is specifically captured by hybridization of PCR product to a target-specific oligonucleotide capture probe that has been bound to the microtiter plate well. The bound product is suitably detected according to the type of label used. For example, if biotin is used as a label, avidin HRP complex is added and is reacted with either (a) hydrogen peroxide substrate and O-phenylene diamine (OPD) chromogen or (b) hydrogen peroxide substrate and tetramethylbenzidine chromogen (TMB). A color metric signal develops, allowing for the quantitative detection of the PCR amplified DNA.

The present invention also relates to kits comprising the primers and probes of the invention. A useful kit can contain probes for detecting chloroplast organelles and kingdom, phylum, class, order, family, and/or genus specific nucleic acid. In some cases, the probes may be fixed to an appropriate support such as for example a microarray chip. The kit can also contain the universal primer pairs of the invention, e.g. for PCR amplification. Other optional components of the kit include, for example, polymerase, the substrate nucleoside triphosphates, means used to label (for example, Taqman fluorescent dyes, avidin-enzyme conjugate and enzyme substrate and chromogen if the label is biotin) or detect label, and the appropriate buffers for PCR, or hybridization reactions, etc. In addition to the above components, the kit can also contain biological sample collection devices, instructions for carrying out amplification and detection methods of the invention, etc.

The invention can be used in environmental monitoring, aimed at detecting changes in biological populations within a defined environment over time. Identification and detection prior to the onset of clinical symptoms can greatly improve the management of an exposed population and facilitate treatment of infected patients, as well as increase the effectiveness of management efforts. The invention can also be applied to monitor organismal levels in air, water or any other natural microbial ecosystem. In addition, the invention can be used to rapidly characterize an unknown biological sample prior to or after culturing.

In an environmental monitoring scenario, biological material in air or water can be collected on filters, beads or other collections media by drawing the sample through the collection media. Genetic material is extracted from collected material and processed by employing real time PCR or immunological assays. A local computer used to collect sample data can be attached to a network and investigators may access and analyze data remotely from their individual laboratories/institutions.

Field samples or laboratory samples can also be analyzed to determine general taxonomic information about the organism or sample of interest.

The primers and probes of the present invention are directly applicable to field monitoring/protection; environmental monitoring (air, water and soil); food testing and safety; biopharmaceutical monitoring, civil preparedness/counterterrorism and laboratory diagnostics and any other application where it is desired to determine whether organisms are present.

Traditional enrichment techniques and the pure culture approach to microbiology have offered only a narrow window into establishing the organismal or microbial diversity of an environment. In order to understand the true organismal diversity of an environment it is necessary to collect and analyze total genetic material from living, non-living, microbial and non-microbial organisms. This invention thus provides a broader display of community structure and allows monitoring of changes over time or an event. It also provides a rapid and sensitive test to detect and classify the presence of all organismal DNA in a sample and to further identify the taxonomic group from which the DNA originates.

In addition, many standard laboratory techniques used for the characterization of microbial organisms in a sample require time-consuming techniques such as gram staining, culturing and phenotypic typing. And many times such phenotypic tests are followed by a suite of genetic tests to further delineate the nature of the unknown sample. The invention will facilitate the initial rapid typing of unknown microbial organisms by providing a rapid PCR test to determine the broad taxonomic character of the organism

In the foregoing and in the following examples, all temperatures are set forth uncorrected in degrees Celsius; and, unless otherwise indicated, all parts and percentages are by weight.

The entire disclosure of all applications, patents and publications, cited above and below, are hereby incorporated by reference.

### EXAMPLE I

Universal primers are designed by the following process:

1. E. coli 16s rDNA sequence is mapped and compared to existing 16s “universal” primers found in the literature.

2 The location of known existing conserved primers located in the sequences are mapped to find areas where two primer regions are separated by a 100-200-nucleotide region.

3. A region at the 3′ end of the 16s molecule is identified having several conserved primer regions. Other potential regions were at the beginning of the sequence (5′ end) and in the 500-600 nucleotide region in the middle of the sequence.

4. The nucleotides mapped at the 3′ end range from nucleotide 1326 (E. coli numbering) to 1542.

5. The forward primer is selected in a commonly used region of the 16s gene (nucleotides 1389-1406).

6. The reverse primer is selected after aligning the 3′ ends of various 16s/18s sequences and visually examining them for regions of conservation. The final region comprises nucleotides 1491-1512.

7. In E. coli this region corresponds to a product size of (1512−1389=123) 123 nucleotides, which is an ideal size for performing real-time/TAQMAN PCR. The 18s rDNA sequence of the yeast, Saccharomyces cerevisiae, corresponds to nucleotides 1626-1643 for the forward primer and 1754-1775 for the reverse, resulting in a size of 149 nucleotides.

8. The primers are prepared and tested with a bacterium (E. coli) and a plant (lettuce).

### EXAMPLE II

A diverse range of organisms tested with the universal primer pair of the present invention in a SYBR-based detection is shown in table 5. Total DNA was extracted from each organism in the list and used in conjunction with the universal forward and universal reverse primers to amplify the conserved region of the 16s (18s) rDNA gene. SYBR-green based detection was used in the universal real-time assay merely to demonstrate that amplification was occurring with the primers. To further demonstrate the linear amplification of DNA across three taxonomic domains (Archea, Bacteria and Eukarya), DNA was extracted from a representative organism from each domain and was used in a dilution series in the universal PCR amplification. The PCR amplification was linear with respect to DNA concentration as can be seen in FIG. 7.

TABLE 5

| Organism | Classification | SYBR |
|---|---|---|
| Escherichia coli | gamma proteobacteria | + |
| Erwinia herbicola | gamma proteobacteria | + |
| Alcaligenes eutrophus | beta proteobacteria | + |
| Bacillus subtilis | bacillus | + |
| Bacillus thuringiensis | bacillus | + |
| Bacillus lichenformis | bacillus | + |
| Bacillus globigii | bacillus | + |
| Bacillus cereus | bacillus | + |
| Enterococcus gallinarum | gm + | + |
| Staphylococcus warneri | gm + | + |
| Streptococcus bovis | gm + | + |
| Streptomyces griseus | actinomycete | + |
| Saccharomyces cerevisiae | yeast | + |
| Aspergillus oryzae | fungus | + |
| Methanosarcian acetivorans | archeaobacteria | + |
| bee pollen | mixed | + |
| poplar pollen | plant | + |
| white birch pollen | plant | + |
| lettuce | plant | + |

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A pair of oligonucleotide primers useful for amplifying a target nucleotide sequence that is substantially conserved among organismal nucleic acids, wherein the forward primer is SEQ ID NO. 1 or a sequence having at least 80% identity to SEQ ID NO. 1 or a sequence capable of hybridizing to SEQ ID NO. 1 under low stringency conditions
and the reverse primer is SEQ ID NO. 2 or a sequence having at least 80% identity to SEQ ID NO. 2 or a sequence capable of hybridizing to SEQ ID NO. 2 under low stringency conditions.

2. An oligonucleotide probe useful for detecting 16s(18s) ribosomal DNA nucleic acid from an organism, of sequence (SEQ ID NO. 3), (SEQ ID NO. 4), (SEQ ID NO. 5), (SEQ ID NO. 6), (SEQ ID NO. 7), (SEQ ID NO. 8), or a sequence fully complementary to one of said sequences.

3. A kit useful for detecting and enumerating total DNA content in a biological sample, comprising a first nucleic acid sequence of SEQ ID NO. 1, and a second nucleic acid sequence of SEQ ID NO. 2.

4. The kit of claim 3, further comprising at least one chloroplast, kingdom, phylum, class, order, or family specific oligonucleotide probe.

5. A kit of claim 4 wherein said probe has the sequence (SEQ ID NO. 3), (SEQ ID NO. 4), (SEQ ID NO. 5), (SEQ ID NO. 6), (SEQ ID NO. 7), (SEQ ID NO. 8), or a sequence fully complementary to one of said sequences.

6. The kit of claim 4, wherein said probe has the sequence (SEQ ID NO. 9), (SEQ ID NO. 10), (SEQ ID NO. 11), (SEQ ID NO. 12), (SEQ ID NO. 13), (SEQ ID NO. 14), (SEQ ID NO. 15), (SEQ ID NO. 16), (SEQ ID NO. 17), (SEQ ID NO. 18), (SEQ ID NO. 19), (SEQ ID NO. 20), (SEQ ID NO. 21), (SEQ ID NO. 22), (SEQ ID NO. 23), or (SEQ ID NO. 24), or a sequence fully complementary to one of said sequences.

7. The kit of claim 4, further comprising a panel of oligonucleotide probes comprising at least two oligonucleotide probes of the sequence (SEQ ID NO. 3), (SEQ ID NO. 4), (SEQ ID NO. 5), (SEQ ID NO. 6), (SEQ ID NO. 7), (SEQ ID NO. 8), (SEQ ID NO. 9), (SEQ ID NO. 10), (SEQ ID NO. 11), (SEQ ID NO. 12), (SEQ ID NO. 13), (SEQ ID NO. 14), (SEQ ID NO. 15), (SEQ ID NO. 16), (SEQ ID NO. 17), (SEQ ID NO. 18), (SEQ ID NO. 19), (SEQ ID NO. 20), (SEQ ID NO. 21), (SEQ ID NO. 22), (SEQ ID NO. 23), (SEQ ID NO. 24), or a sequence fully complementary to one of said probes.

8. An oligonucleotide probe for detecting 16s(18s) ribosomal DNA from an organism which has the sequence (SEQ ID NO. 3), (SEQ ID NO. 4), (SEQ ID NO. 5), (SEQ ID NO. 6), (SEQ ID NO. 7), (SEQ ID NO. 8), (SEQ ID NO. 9), (SEQ ID NO. 10), (SEQ ID NO. 11), (SEQ ID NO. 12), (SEQ ID NO. 13), (SEQ ID NO. 14), (SEQ ID NO. 15), (SEQ ID NO. 16), (SEQ ID NO. 17), (SEQ ID NO. 18), (SEQ ID NO. 19), (SEQ ID NO. 20), (SEQ ID NO. 21), (SEQ ID NO. 22), (SEQ ID NO. 23), (SEQ ID NO. 24), or a sequence fully complementary to one of said sequences.

9. A method for detecting total organismal nucleic acid contained in a sample, comprising amplifying a region of nucleic acid(s) from a 16s(18s) ribosomal DNA gene, wherein the amplification is achieved by a polymerase chain reaction using a pair of primers having the sequence (SEQ ID NO. 1) and (SEQ ID NO. 2) or a sequence having at least 80% identity to one of said sequences or capable of hybridizing to said sequences under low stringency conditions.

10. A method of claim 9 further comprising detecting amplified DNA specific to a certain organism by probing said amplified DNA using an oligonucleotide probe which is of the sequence (SEQ ID NO. 3), (SEQ ID NO. 4), (SEQ ID NO. 5), (SEQ ID NO. 6), (SEQ ID NO. 7), (SEQ ID NO. 8), (SEQ ID NO. 9), (SEQ ID NO. 10), (SEQ ID NO. 11), (SEQ ID NO. 12), (SEQ ID NO. 13), (SEQ ID NO. 14), (SEQ ID NO. 15), (SEQ ID NO. 16), (SEQ ID NO. 17), (SEQ ID NO. 18), (SEQ ID NO. 19), (SEQ ID NO. 20), (SEQ ID NO. 21), (SEQ ID NO. 22), (SEQ ID NO. 23), (SEQ ID NO. 24), or a sequence fully complementary to one of said sequences.

11. A method of claim 10, wherein at least two of said probes are used.

12. A process according to claim 9, further comprising separating PCR products of different sequence composition by TGGE or DGGE analysis.

13. The process of claim 9, wherein said probe is labeled at its 5′ end by a reporter dye and at its 3′ end by a molecule capable of quenching said reporter dye.

14. A method for determining total DNA content in a sample, comprising amplifying a target nucleotide sequence using at least two primers complementary to sequences which are present in substantially all organismal species.

15. A method of claim 14 wherein said amplification is for a time and under conditions sufficient to generate a level of an amplification product which is proportional to the level of organismal DNA in said sample.

16. A method according to claim 14 wherein said target nucleotide sequence is DNA, rRNA or rDNA.

17. A method according to claim 16 wherein the rDNA is 16s(18s) rDNA.

18. A method according to claim 17 wherein the target sequence comprises a sequence specific for an organism to be identified or which is associated with a kingdom, phylum, class, order or family.

19. A method according to claim 14 wherein the sample is a biological, medical, agricultural, industrial or environmental sample which is a liquid, solid, slurry, air, vapor, droplet, aerosol or a combination thereof.

20. A method according to claim 19 wherein the sample is from soil, water, a hot mineral spring, plant, the Antarctic, air, extraterrestrial origin, an industrial site, a waste site, a waste stream, an area of an oil spill or aromatic or complex molecule contamination or pesticide contamination, or is an aquatic or a biopharmaceutical product; or wherein the sample comprises food, a food component, a food derivative, a food ingredient, a food product formed in the dairy industry, or a combination thereof.

21. A method according to claim 19 wherein the amplification and probe detection is by Real-Time PCR.

22. A method according to 14 wherein the organismal DNA is amplified with a primer pair comprising a forward primer having the sequence set forth in SEQ ID NO: I or a sequence having at least about 80% identity thereto or a sequence capable of hybridizing to SEQ ID NO: 1 or its complementary form under low stringency conditions.

23. A method according to claim 22 wherein the forward primer comprises the sequence set forth in SEQ ID NO: 1.

24. A method according to claim 14 wherein the organismal DNA is amplified with a primer pair comprising a reverse primer having the sequence set forth in SEQ ID NO:2
or a sequence having at least about 80% identity thereto or a sequence capable of hybridizing to SEQ ID NO:2 or its complementary form under low stringency conditions.

25. A method according to claim 23 wherein the reverse primer comprises the sequence set forth in SEQ ID NO:2.

26. A method according to claim 14 wherein the amplified product is assayed using a labeled probe having a sequence (SEQ ID NO. 3), (SEQ ID NO. 4), (SEQ ID NO.5), (SEQ ID NO. 6), (SEQ ID NO. 7), (SEQ ID NO. 8), (SEQ ID NO. 9), (SEQ ID NO. 10), (SEQ ID NO. 11), (SEQ ID NO. 12), (SEQ ID NO. 13), (SEQ ID NO. 14), (SEQ ID NO. 15), (SEQ ID NO. 16), (SEQ ID NO. 17), (SEQ ID NO. 18), (SEQ ID NO. 19), (SEQ ID NO. 20), (SEQ ID NO. 21), (SEQ ID NO. 22), (SEQ ID NO. 23), (SEQ ID NO. 24), or a sequence fully complementary to one of said sequences.

27. A method for identifying and classifying an organism in a sample, said method comprising amplifying DNA in said sample using the method of claim 22, and assaying said amplified DNA with a probe which is either specific for an organism to be identified or which is associated with a kingdom, phylum, class, order or family specific probe.

28. A method according to claim 27 wherein the amplified DNA is 16s(18s) rDNA.

29. A method according to claim 27 wherein the kingdom-specific probe is also a phylum, class, order or family-specific probe.

30. A method according to claim 27 wherein said target nucleotide sequence is DNA.

31. A method according to claim 30 wherein said target nucleotide sequence is 16s(18s) rDNA.

32. A combination comprising an oligonucleotide of (SEQ. ID NO 1) or a sequence having at least 80% identity to (SEQ. ID NO 1) or a sequence capable of hybridizing to (SEQ ID NO. 1) under low stringency conditions and
an oligonucleotide of (SEQ. ID NO 2), or a sequence having at least 80% identity to (SEQ. ID NO 2) or a sequence capable of hybridizing to (SEQ ID NO. 2) under low stringency conditions.

33. A method according to claim 14 wherein the amplified product is assayed using a labeled probe having a fragment or variant of sequence of SEQ ID NO: 3, 4, 5, 6, 7, 8, 9, 10, 11, 12, 13, 14, 15, 16, 17, 18, 19, 21, 22, 23, 24.
